(19) United States
(12) Patent Application Publication
(10) Pub. No.: US 2023/0181135 A1
(43) Pub. Date: Jun. 15, 2023

(54) RADIOGRAPHIC IMAGING DEVICE AND RADIOGRAPHY SYSTEM

(71) Applicant: Konica Minolta, Inc., Tokyo (JP)

(72) Inventors: Tadashi MARUYAMA, Tokyo (JP); Hiroshi UTSUNOMIYA, Tokyo (JP)

(21) Appl. No.: 18/079,257

(22) Filed: Dec. 12, 2022

(30) Foreign Application Priority Data
Dec. 14, 2021 (JP) 2021-202134

(51) Int. Cl. A61B 6/00

(52) U.S. Cl. CPC A61B 6/4283; A61B 6/54; A61B 6/44

(57) ABSTRACT

A radiographic imaging device that obtains a radiographic image, includes a battery, a first hardware processor, and a storage. The battery drives the radiographic imaging device. The first hardware processor measures an amount of power remaining in the battery. The storage stores a first threshold and a second threshold of the amount of power remaining in the battery. The first threshold is used to allow photographing of a first photography mode. The second threshold is used to allow photographing of a second photography mode that consumes less power than the first photography mode.

### CROSS-REFERENCE TO RELATED APPLICATIONS

The present invention claims priority under 35 U.S.C. §119 to Japanese Patent Application No. 2021-202134 filed on Dec. 14, 2021, the entire content of which is incorporated herein by reference.

### BACKGROUND

### Technological Field

The present invention relates to a radiographic imaging device and a radiography system.

### Description of the Related Art

Radiographic imaging devices using a flat panel detector (FPD) are conventionally known, and in recent years, mobile and portable (cassette) radiographic imaging devices that contain radiation detecting elements in their cases have been used. Many portable radiographic imaging devices are driven by a built-in battery.

A battery-operated FPD may run out and be powered down in the middle of photography operation. In this case, photographing must be performed again, which causes an increase in burden on a subject (patient) and a decrease in operation efficiency. In order to avoid such a situation, only when remaining battery power is still sufficient to perform photographing once, the state of being capable of photographing is notified to a user, and photographing is then started.

In one example, a radiographic imaging system that has been developed prohibits emission of radiation from a photographing apparatus in a state in which remaining battery power is less than a predetermined threshold at the time an FPD receives a photographing start signal (refer to JP 5325571B).

In another example, a movable radiographic apparatus that has been developed acquires a plurality of pieces of examination order information and determines whether each photographing is moving image photography or still image photography, and it derives a photographing order that does not cause a lack of battery power of an FPD (refer to JP 6668716B).

### SUMMARY

Unfortunately, the technique disclosed in JP 5325571B uses only one threshold that is set to prohibit photography with respect to the remaining battery power, but the consumption of power required to perform photographing once varies depending on photography mode. For example, the consumption of power that is required to perform moving image photographing once is greater than that for still image photographing. Thus, setting a threshold of remaining battery power so as to enable moving image photography reduces the number of photographic images that can be obtained in still image photography, to be smaller than before.

As illustrated in FIG. 13, a threshold for prohibiting photographing may be set to a consumption of power required to perform photographing once in a photography mode that consumes a large amount of power (high power consumption photography). In this case, although a consumption of power required to perform photographing once in a photography mode that consumes a small amount of power (low power consumption photography) is lower than the set threshold, the low power consumption photography is disabled. That is, in the state in which the remaining battery power is insufficient to perform the high power consumption photography, all types of photography are prohibited in existing techniques. However, it is desirable to continue photography if the low power consumption photography is available.

The technique disclosed in JP 6668716B is designed to change the photographing order, and therefore, photographing is not performed in the order as originally desired by a user.

The present invention has been made in view of the above-described problems of existing techniques, and an object thereof is to enable low power consumption photography although remaining battery power is insufficient to perform high power consumption photography.

To achieve at least one of the abovementioned objects, according to an aspect of the present invention, a radiographic imaging device reflecting one aspect of the present invention is a radiographic imaging device that obtains a radiographic image, including:
a battery that drives the radiographic imaging device;
a first hardware processor that measures an amount of power remaining in the battery; and
a storage that stores a first threshold and a second threshold of the amount of power remaining in the battery, the first threshold used to allow photographing of a first photography mode, the second threshold used to allow photographing of a second photography mode that consumes less power than the first photography mode.

To achieve at least one of the abovementioned objects, according to another aspect of the present invention, a radiography system reflecting one aspect of the present invention is a radiography system including the radiographic imaging device and the control device, and the control device includes a second hardware processor that notifies the result of determination, based on the information related to the result of determination, which is received from the radiographic imaging device.

To achieve at least one of the abovementioned objects, according to another aspect of the present invention, a radiography system reflecting one aspect of the present invention is a radiography system including:
a radiographic imaging device that obtains a radiographic image; and
a control device that controls radiography performed by the radiographic imaging device, the radiographic imaging device including:
a battery that drives the radiographic imaging device; and
a first hardware processor that measures an amount of power remaining in the battery and transmits information of the amount of power remaining in the battery to the control device,
the control device including:
a storage that stores a first threshold and a second threshold of the amount of power remaining in the battery, the first threshold used to allow photographing of a first photography mode, the second threshold used to allow photographing of a second photography mode that consumes less power than the first photography mode; and
a second hardware processor that:
determines that photographing of the first photography mode is available in a case in which the amount of power remaining in the battery is equal to or greater than the first threshold;
determines that photographing of the second photography mode is available but photographing of the first photography mode is unavailable in a case in which the amount of power remaining in the battery is equal to or greater than the second threshold but is less than the first threshold;
determines that photographing of the second photography mode is unavailable in a case in which the amount of power remaining in the battery is less than the second threshold; and
notifies result of the determination.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 illustrates an example of the whole configuration of a radiography system of a first embodiment of the present invention;

FIG. 2 is a block diagram illustrating a functional configuration of a main body;

FIG. 3 is a block diagram illustrating a functional configuration of an FPD cassette;

FIG. 4 illustrates examples of a first photography mode and a second photography mode;

FIG. 5 illustrates available photography modes corresponding to remaining battery power;

FIG. 6 is a flowchart illustrating a first process of determining remaining battery power, which is executed by the FPD cassette;

FIGS. 7A to 7C illustrate battery state signs of the FPD cassette;

FIG. 8 is an example of an operation screen displayed on a display of the main body;

FIG. 9 is an example of a battery state sign that shows the state of being capable of performing each of high power consumption photography and low power consumption photography, which is displayed on the display of the main body;

FIG. 10 is an example of the battery state sign that shows the state of prohibiting the high power consumption photography but allowing the low power consumption photography, which is displayed on the display of the main body;

FIG. 11 is an example of the battery state sign that shows the state of prohibiting both of the high power consumption photography and the low power consumption photography, which is displayed on the display of the main body;

FIG. 12 is a flowchart illustrating a second process of determining remaining battery power, which is executed by the main body of a second embodiment; and

FIG. 13 illustrates available photography modes corresponding to remaining battery power in a conventional technique.

### DETAILED DESCRIPTION OF THE EMBODIMENTS

Hereinafter, embodiments of the present invention will be described with reference to the drawings. However, the scope of the invention is not limited to the disclosed embodiments or illustrated examples.

### <First Embodiment>

### (Configuration of Radiography System)

First, a configuration of a first embodiment will be described.

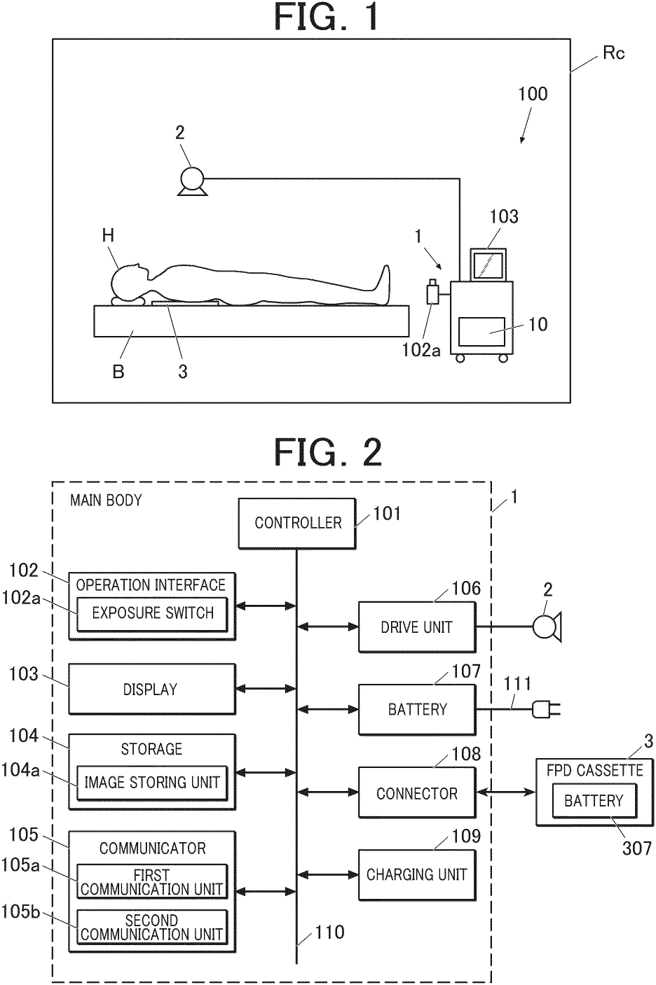

FIG. 1 illustrates an example of the whole configuration of a radiography system 100 of the first embodiment.

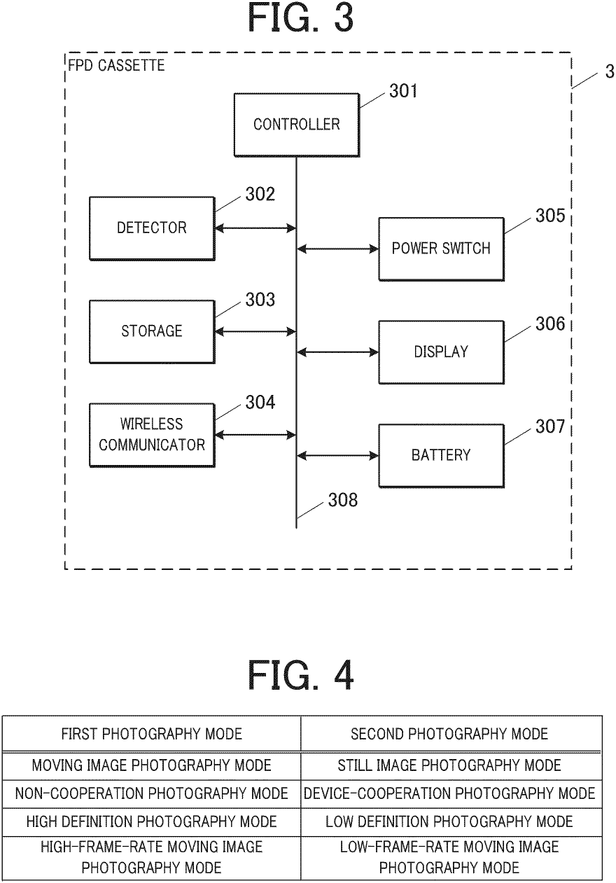

The radiography system 100 is a system for performing radiography on a patient, such as a patient who has a difficulty in moving, by visiting the patient in a ward. The radiography system 100 includes a main body 1, a radiation source 2, and an FPD cassette 3. The radiography system 100 has wheels on the main body 1 and is constructed as an instrument carriage that can be moved. The main body 1 is provided with a storage space 10 for storing the FPD cassette 3. The storage space 10 is provided with a connector 108 (refer to FIG. 2) for connecting the stored FPD cassette 3, and thus, the stored FPD cassette 3 can be carried while a battery 307 (refer to FIGS. 2 and 3) of the FPD cassette 3 is being charged.

The main body 1 has functions of a console (control device) that performs wireless communication with the FPD cassette 3, confirmation of an obtained photographic image, and control related to photography.

Although an example of using the radiography system 100 as an instrument carriage is described herein, use of the radiography system 100 is not limited to photography during visit to a patient, and the radiography system 100 can also be used in wireless photography in a general imaging room.

As illustrated in FIG. 1, the radiography system 100 is brought into an operating room, an intensive care unit, a hospital room Rc, or the like, and the FPD cassette 3 is set up. For example, the FPD cassette 3 is inserted between a bed “B” and a subject “H” (examinee) lying on the bed “B” or into a slot (not illustrated) that is provided on a side opposite to the subject “H” on the bed “B.” In such a state, the radiography system 100 performs still image photography or moving image photography on the subject “H” by emitting radiation from the radiation source 2. The still image photography is to obtain one image of a subject in response to one photography operation (pressing down an exposure switch 102a). The moving image photography is to acquire a moving image by continuously obtaining a plurality of images of a subj ect in response to one photography operation. The moving image includes a moving image that is composed of a predetermined number of photographed frames, and fluoroscopic images such as of tomosynthesis performed by photographing while radiation is emitted. The moving image photography is performed by repeatedly emitting pulses of radiation, such as X-rays, to a subject at a predetermined time interval (pulse irradiation), or continuously emitting radiation of a low dose rate without interruption (continuous irradiation).

FIG. 2 is a block diagram illustrating a functional configuration of the main body 1 of the radiography system 100.

As illustrated in FIG. 2, the main body 1 includes a controller 101 (second hardware processor), an operation interface 102, a display 103, a storage 104, a communicator 105, a drive unit 106, a main body battery 107, a connector 108, and a charging unit 109 that are connected to each other via a bus 110.

The controller 101 is composed of a central processing unit (CPU), a random access memory (RAM), and so on. The CPU of the controller 101 reads a system program and various processing programs, which are stored in the storage 104, in response to input from the operation interface 102, and the CPU loads them into the RAM to execute various processes in accordance with the loaded programs.

The operation interface 102 includes a touch panel in which transparent electrodes are arranged in a grid pattern so as to cover the surface of the display 103. The operation interface 102 detects a position that is pressed down by a finger, a stylus, or the like, and it then inputs information of this position to the controller 101, as operation information.

The operation interface 102 also includes an exposure switch 102a that is used by a user to instruct start of emitting radiation.

The display 103 is composed of a monitor, such as a liquid crystal display (LCD), and performs displaying in accordance with an instruction of a display signal input from the controller 101.

The storage 104 is composed of a non-volatile semiconductor memory, a hard disk drive, and so on. The storage 104 stores various programs that are executed by the controller 101, parameters necessary for programs to execute processes, and data such as result of processing.

The storage 104 is provided with an image storing unit 104a. The image storing unit 104a provides a memory area that temporarily stores image data, which is obtained in photography during visit to a patient, before the image data is transferred to an external device such as an analysis device or a picture archiving and communication system (PACS).

The communicator 105 includes a first communication unit 105a and a second communication unit 105b. The first communication unit 105a transmits and receives data to and from the FPD cassette 3 by wireless communication. The type of wireless communication used by the first communication unit 105a is near field communication (NFC), Bluetooth, Wi-Fi (registered trademark), or the like. The second communication unit 105b transmits and receives data to and from an RIS, an analysis device, a PACS, or other external device, which is connected to a communication network (in-hospital network), such as a local area network (LAN) or a wide area network (WAN), via a wireless access point provided at each position in a hospital.

The drive unit 106 is a circuit that drives a tubular lamp of the radiation source 2. The drive unit 106 and the radiation source 2 are connected to each other via a cable.

The main body battery 107 supplies power to each component of the main body 1 and to the radiation source 2. The main body battery 107 can be charged from the outside via an AC cable 111. The main body battery 107 is charged in advance via the AC cable 111 during a time when photography operation is not performed. The AC cable 111 is contained inside the main body 1 at the time of carrying.

The connector 108 is provided inside the storage space 10 and is electrically connected to the FPD cassette 3 stored in the storage space 10. The FPD cassette 3 may perform data communication via the connector 108 while being connected to the connector 108.

The charging unit 109 is a circuit for charging the battery 307 of the FPD cassette 3 that is connected via the connector 108, with electric power supplied from the main body battery 107, based on control from the controller 101 during a time of not performing photography.

The radiation source 2 is a radiation irradiation device that is driven by the drive unit 106 and that emits radiation (X-rays) to a subject “H.”

The FPD cassette 3 is a radiographic imaging device that obtains a radiographic image and generates a radiographic image (image data) based on radiation emitted from the radiation source 2. The FPD cassette 3 is a portable radiation detector that has the rechargeable battery 307 as a drive source, and it can be used in still image photography and in moving image photography.

FIG. 3 is a block diagram illustrating a functional configuration of the FPD cassette 3.

As illustrated in FIG. 3, the FPD cassette 3 includes a controller 301 (first hardware processor), a detector 302, a storage 303, a wireless communicator 304, a power switch 305, a display 306, and a battery 307 that are connected to each other via a bus 308.

The controller 301 is composed of a CPU, a RAM, and so on. The CPU of the controller 301 reads various processing programs, which are stored in the storage 303, and the CPU loads them into the RAM to execute various processes in accordance with the loaded programs.

The controller 301 controls the detector 302 to read image signals that are detected by the detector 302, and it then generates image data of a radiographic image based on these image signals and transmits the generated image data to the main body 1 via the wireless communicator 304.

The detector 302 includes a glass substrate on which a plurality of detecting elements are two-dimensionally arranged at predetermined positions. Each of the detecting elements detects radiation that has been emitted from the radiation source 2 and has then passed through at least a subject “H,” in accordance with the intensity of the radiation. The detecting element then converts the detected radiation into an electrical signal and accumulates it. Each of the detecting elements is composed of a semiconductor image sensor, such as a photodiode. Each of the detecting elements is connected to a switching device, such as a thin film transistor (TFT), and the switching device controls accumulation and reading of an electrical signal, whereby image data is obtained.

There are an indirect conversion FPD and a direct conversion FPD. The indirect conversion FPD converts radiation into an electrical signal via a scintillator by using a photoelectric conversion element. The direct conversion FPD directly converts radiation into an electrical signal. Either the indirect conversion FPD or the direct conversion FPD can be used as the FPD cassette 3.

The storage 303 is composed of a non-volatile semiconductor memory and so on. The storage 303 stores various programs that are executed by the controller 301, parameters necessary for programs to execute processes, and data such as result of processing.

The storage 303 stores a first threshold and a second threshold of the amount of power remaining in the battery 307 (remaining battery power). The first threshold allows photographing of a first photography mode (high power consumption photography). The second threshold allows photographing of a second photography mode that consumes less power than the first photography mode (low power consumption photography). That is, the first threshold is greater than the second threshold.

The first threshold is a limit value that determines whether the high power consumption photography is available and whether to allow or prohibit the high power consumption photography.

The second threshold is a limit value that determines whether the low power consumption photography is available and whether to allow or prohibit the low power consumption photography.

The first threshold and the second threshold are specified in accordance with the photography modes that are employed as the first photography mode and the second photography mode.

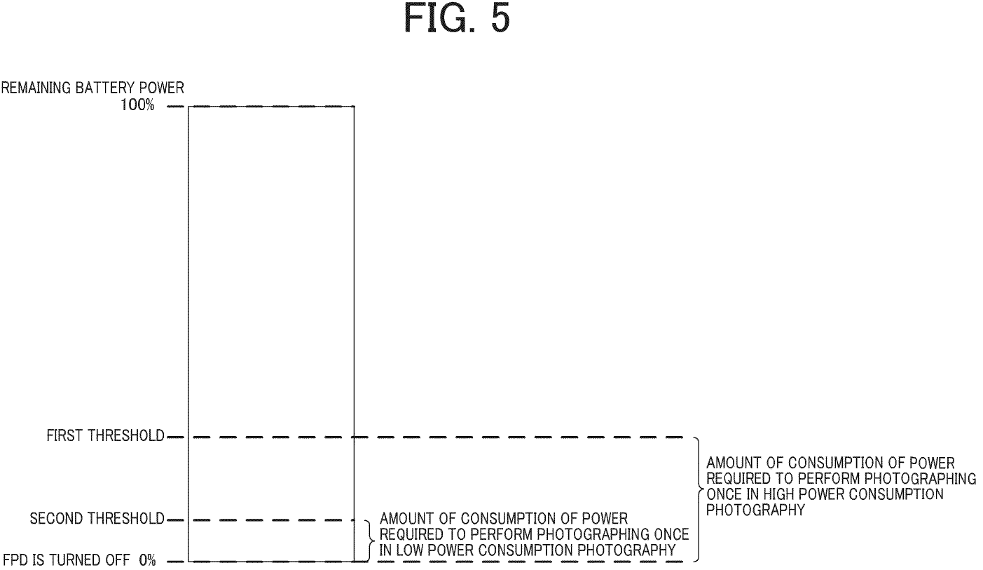

FIG. 4 illustrates examples of the first photography mode and the second photography mode.

In one example, the first photography mode is a moving image photography mode, whereas the second photography mode is a still image photography mode.

The first photography mode is also a photography mode in which photographing is performed without cooperation with the radiation source 2 (non-cooperation photography mode), whereas the second photography mode is a photography mode in which photographing is performed in cooperation with the radiation source 2 (device-cooperation photography mode).

The non-cooperation photography mode is a photography mode in which the FPD cassette 3 itself detects emission of radiation and starts photographing. That is, in the non-cooperation photography mode, photographing is performed without notifying timing of emission of radiation to the FPD cassette 3 from the main body 1 that controls driving of the radiation source 2. In the non-cooperation photography mode, the FPD cassette 3 should monitor whether radiation is emitted and thus consumes more power than when it is in the device-cooperation photography mode.

The device-cooperation photography mode is a photography mode in which the FPD cassette 3 and the main body 1, which controls driving of the radiation source 2, perform photographing in synchronization with each other. For example, the FPD cassette 3 receives information of the time that the radiation source 2 starts emission of radiation, from the main body 1, and it starts photographing upon receiving this information. In the device-cooperation photography mode, photographing is performed while the first communication unit 105a of the main body 1 and the wireless communicator 304 of the FPD cassette 3 transmit and receive information related to timing to and from each other.

The first photography mode is also a high definition photography mode, whereas the second photography mode is also a low definition photography mode with a resolution lower than that of the high definition photography mode.

The high definition photography mode is a photography mode in which the resolution is high and the number of pixels is large compared with the low definition photography mode.

The low definition photography mode is a photography mode in which the resolution is low and the number of pixels is small compared with the high definition photography mode. The low definition photography mode may be a normal photography mode.

In addition, the first photography mode is a high-frame-rate moving image photography mode, whereas the second photography mode is a low-frame-rate moving image photography mode in which the frame rate is lower than that of the high-frame-rate moving image photography mode.

Available photography modes corresponding to remaining battery power will be described with reference to FIG. 5.

The first threshold corresponds to the amount of consumption of power required to perform photographing once in the first photography mode (high power consumption photography). Thus, in the case in which the remaining battery power is equal to or greater than the first threshold, the high power consumption photography is allowed. On the other hand, in the case in which the remaining battery power is less than the first threshold, the high power consumption photography is prohibited.

The second threshold corresponds to the amount of consumption of power required to perform photographing once in the second photography mode (low power consumption photography). Thus, in the case in which the remaining battery power is less than the first threshold but is equal to or greater than the second threshold, the low power consumption photography is allowed. On the other hand, in the case in which the remaining battery power is less than the second threshold, both of the high power consumption photography and the low power consumption photography are prohibited.

When the remaining battery power finally reaches 0%, the power supply of the FPD cassette 3 is turned off.

The wireless communicator 304 transmits and receives data to and from the main body 1 by wireless communication. The type of wireless communication used by the wireless communicator 304 is NFC, Bluetooth, Wi-Fi, or the like.

The power switch 305 is a switch for turning on/off the power supply of the FPD cassette 3.

The display 306 is composed of an LCD or the like and performs displaying in accordance with an instruction of a display signal input from the controller 301. The display 306 may be composed of light emitting diodes (LED) of each color.

The battery 307 is a power storage device that functions as a driving source of the FPD cassette 3 (radiographic imaging device), and it supplies power to each component of the FPD cassette 3.

The controller 301 measures the amount of power remaining in the battery 307. That is, the controller 301 functions as a remaining-battery-power measurement unit.

The controller 301 determines that photographing of the first photography mode is available in the case in which the remaining battery power is equal to or greater than the first threshold. The controller 301 also determines that photographing of the second photography mode is available but photographing of the first photography mode is unavailable in the case in which the remaining battery power is equal to or greater than the second threshold but is less than the first threshold. The controller 301 also determines that photographing of the second photography mode is unavailable in the case in which the remaining battery power is less than the second threshold. That is, the controller 301 also functions as a determination unit.

The first threshold is greater than the second threshold, and therefore, photographing of the second photography mode is also available in the case in which the remaining battery power is equal to or greater than the first threshold. In the case in which the remaining battery power is equal to or greater than the first threshold, the controller 301 allows photographing of the first photography mode and photographing of the second photography mode.

Conversely, the second threshold is smaller than the first threshold, and therefore, photographing of the first photography mode is also unavailable in the case in which the remaining battery power is less than the second threshold. In the case in which the remaining battery power is less than the second threshold, the controller 301 prohibits photographing of the first photography mode and photographing of the second photography mode.

In the case in which the remaining battery power is equal to or greater than the second threshold but is less than the first threshold, the controller 301 allows photographing of the second photography mode but prohibits photographing of the first photography mode.

The controller 301 makes the display 306 display information related to result of determining whether photographing of each photography mode is available, based on the remaining battery power. The controller 301 makes the display 306 show a different display depending on the determination result. That is, the controller 301 also functions as a display controller.

It is enough that the information related to the determination result contains result of determination using the two thresholds (the first threshold and the second threshold) for the remaining battery power. For example, the information may show whether photographing of each photography mode is available, or the information may show the range that the remaining battery power is in, among a range of equal to or greater than the first threshold, a range of equal to or greater than the second threshold but less than the first threshold, and a range of less than the second threshold.

The controller 301 transmits the information related to the determination result to the main body 1, which is a control device for controlling radiography performed by the FPD cassette 3, via the wireless communicator 304. That is, the controller 301 also functions as a transmission controller.

The controller 101 of the main body 1 notifies the determination result, based on the information related to the determination result (such as whether photographing of each photography mode is available) received from the FPD cassette 3. That is, the controller 101 functions as a notification unit.

### (Operation of Radiography System)

Next, operation of the radiography system 100 of the first embodiment will be described.

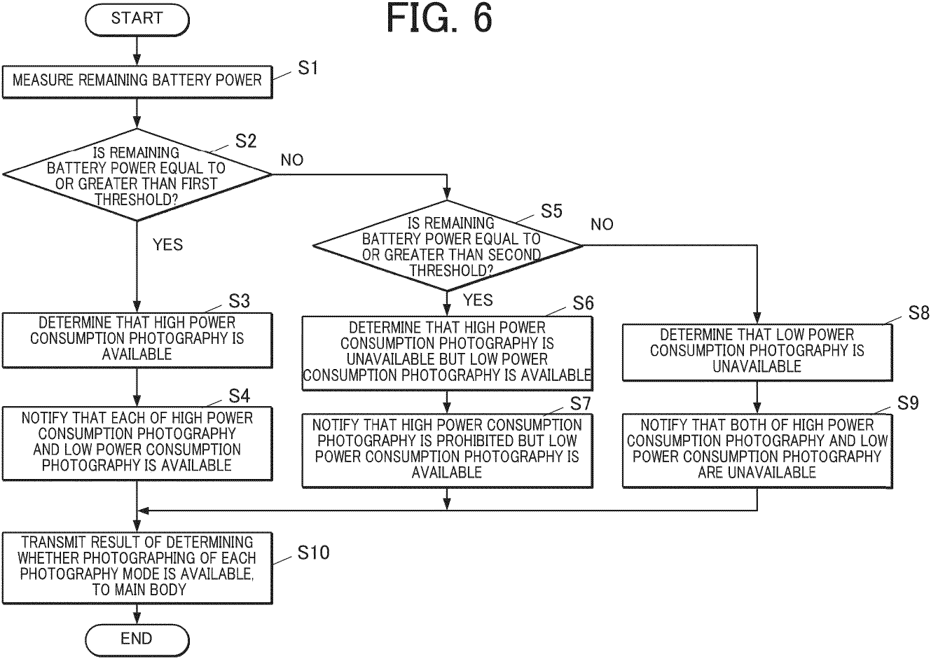

FIG. 6 is a flowchart illustrating a first process of determining remaining battery power, which is executed by the controller 301 of the FPD cassette 3. The first process of determining remaining battery power is performed, such as when the FPD cassette 3 is turned on by the power switch 305, or when photographing is finished once. This process is executed by cooperation of the controller 301 and the program stored in the storage 303.

First, the controller 301 of the FPD cassette 3 measures the remaining battery power (amount of stored power) in the battery 307 (step S1).

Next, the controller 301 determines whether the measured remaining battery power is equal to or greater than the first threshold, which is stored in the storage 303 (step S2).

In the case in which the remaining battery power is equal to or greater than the first threshold (step S2; YES), the controller 301 determines that photographing of the first photography mode (high power consumption photography) is available (step S3). The first threshold is greater than the second threshold, and therefore, photographing of the second photography mode (low power consumption photography) is also available in the case in which the remaining battery power is equal to or greater than the first threshold.

Then, the controller 301 notifies the state that each of the high power consumption photography and the low power consumption photography is available (step S4).

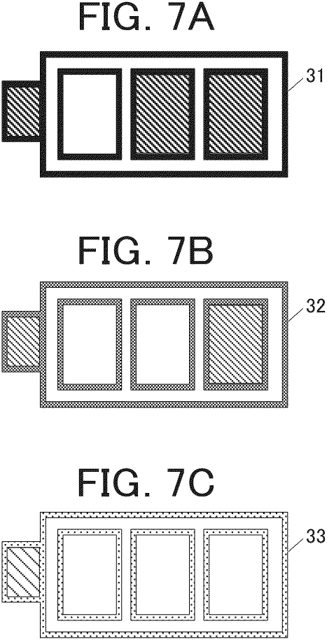

Specifically, the controller 301 makes the display 306 show a battery state sign 31, which is illustrated in FIG. 7A. The battery state sign 31 shows that the remaining battery power is sufficient for both of the high power consumption photography and the low power consumption photography, and for example, it is displayed in blue color.

In the case in which the remaining battery power is less than the first threshold in step S2 (step S2; NO), the controller 301 determines whether the remaining battery power is equal to or greater than the second threshold, which is stored in the storage 303 (step S5).

In the case in which the remaining battery power is equal to or greater than the second threshold (step S5; YES), the controller 301 determines that the high power consumption photography is unavailable but the low power consumption photography is available (step S6).

Then, the controller 301 notifies the state that the high power consumption photography is prohibited but the low power consumption photography is available (step S7). Specifically, the controller 301 makes the display 306 show a battery state sign 32, which is illustrated in FIG. 7B. The battery state sign 32 shows that the remaining battery power is insufficient for the high power consumption photography but is sufficient for the low power consumption photography, and for example, it is displayed in yellow color.

In the case in which the remaining battery power is less than the second threshold in step S5 (step S5; NO), the controller 301 determines that the low power consumption photography is unavailable (step S8). The second threshold is smaller than the first threshold, and therefore, the high power consumption photography is also unavailable in the case in which the remaining battery power is less than the second threshold.

Then, the controller 301 notifies the state that both of the high power consumption photography and the low power consumption photography are prohibited (step S9). Specifically, the controller 301 makes the display 306 show a battery state sign 33, which is illustrated in FIG. 7C. The battery state sign 33 shows that the remaining battery power is insufficient for both of the high power consumption photography and the low power consumption photography, and for example, it is displayed in red color.

As illustrated in FIGS. 7A to 7C, the controller 301 makes the display 306 show a different display by changing the displaying method and the color of the sign of the remaining battery power in accordance with the result of determining whether photographing of each photography mode is available.

After step S4, S7, or S9 is performed, the controller 301 transmits the result of determining whether photographing of each photography mode is available, to the main body 1 via the wireless communicator 304 (step S10).

Then, the first process of determining remaining battery power is completed.

In the main body 1, upon receiving the result of determining whether photographing of each photography mode is available, from the FPD cassette 3 via the first communication unit 105a, the controller 101 makes a notification in accordance with the determination result. Specifically, the controller 101 makes the display 103 show the determination result.

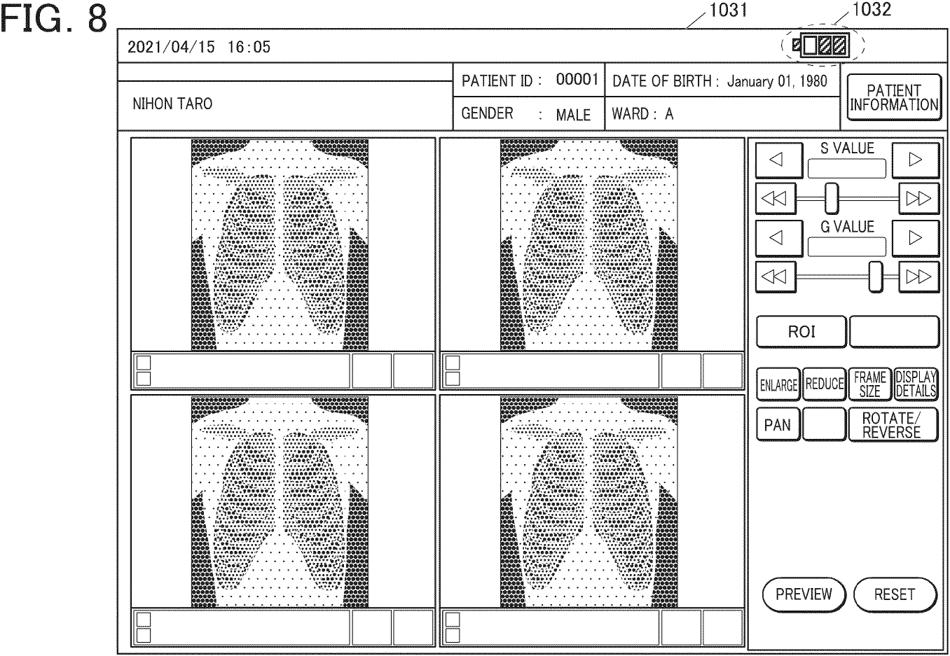

FIG. 8 illustrates an example of an operation screen 1031 displayed on the display 103. The operation screen 1031 contains a panel state display area 1032 at an upper part. The panel state display area 1032 shows the result of determining whether each type of photography is available, which is performed by the FPD cassette 3.

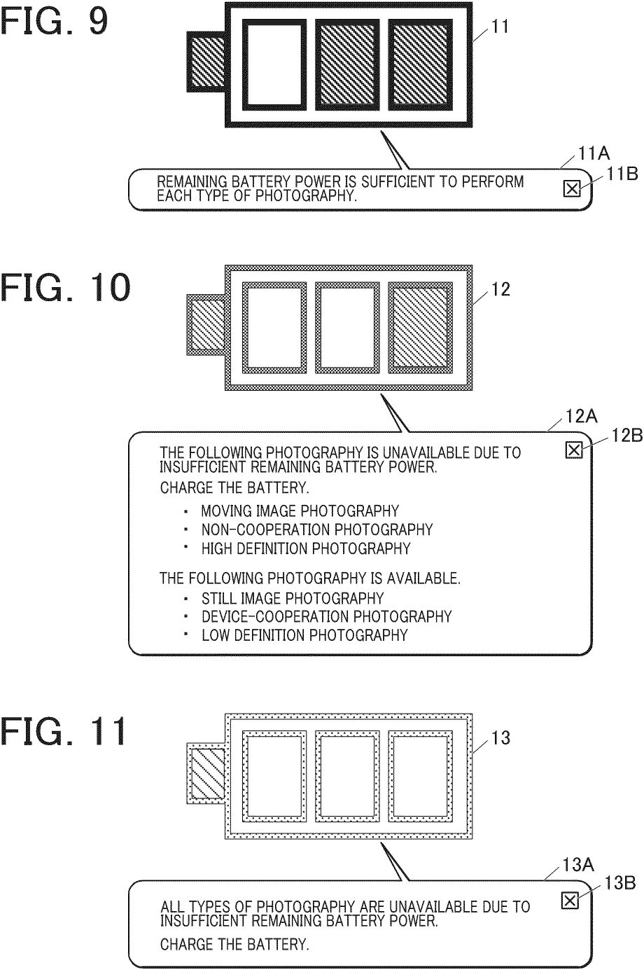

FIGS. 9 to 11 are examples of showing the determination result displayed in the panel state display area 1032.

FIG. 9 is a display example of the battery state sign 11, which shows that the remaining battery power of the FPD cassette 3 is equal to or greater than the first threshold. The battery state sign 11 shows that each of the high power consumption photography and the low power consumption photography is available, and for example, it is displayed in blue color.

In addition to the battery state sign 11, the controller 101 may make the display 103 also display a message 11A: “Remaining battery power is sufficient to perform each type of photography.” In this case, in response to pressing down a close button 11B by operation to the operation interface 102, the controller 101 hides the message 11A.

FIG. 10 is a display example of the battery state sign 12, which shows that the remaining battery power of the FPD cassette 3 is equal to or greater than the second threshold but is less than the first threshold. The battery state sign 12 shows that the high power consumption photography is prohibited but the low power consumption photography is available, and for example, it is displayed in yellow color. In addition to the battery state sign 12, the controller 101 may make the display 103 also display a message 12A that shows photography modes being unavailable due to insufficient remaining battery power as well as photography modes being still available by using the current remaining battery power. In this case, in response to pressing down a close button 12B by operation to the operation interface 102, the controller 101 hides the message 12A.

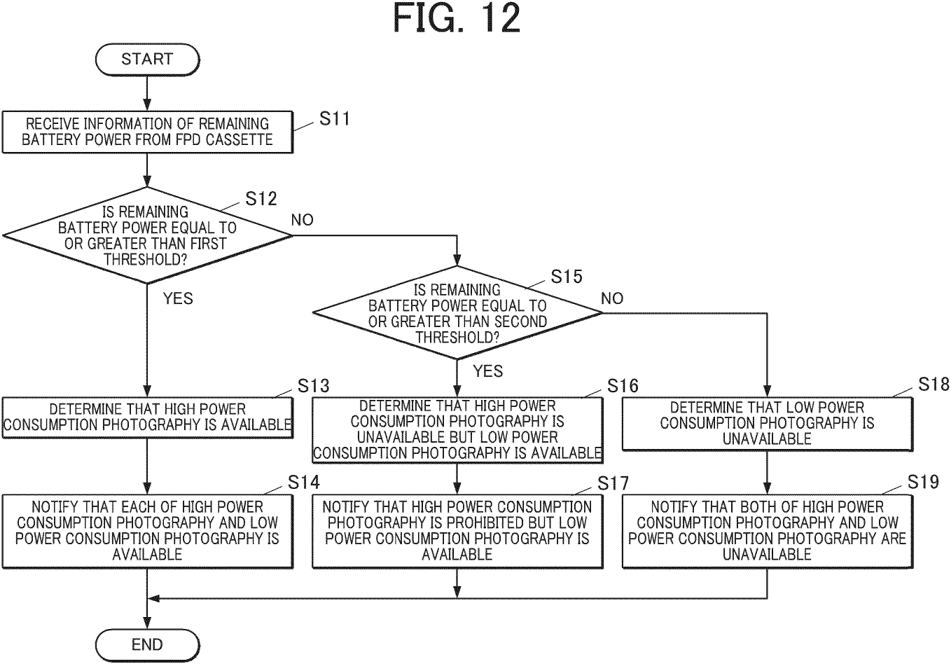

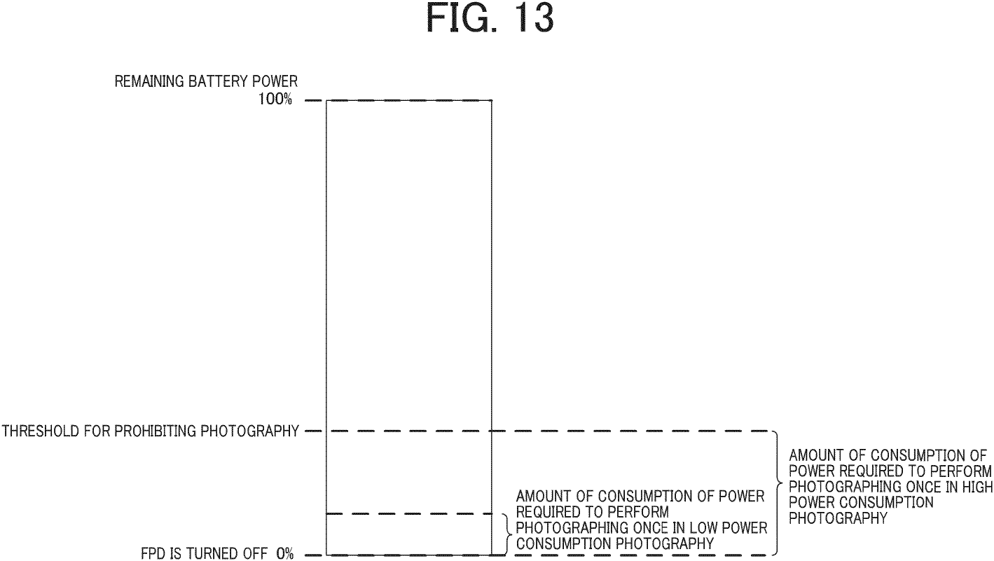

FIG. 11 is a display example of the battery state sign 13, which shows that the remaining battery power of the FPD cassette 3 is less than the second threshold. The battery state sign 13 shows that both of the high power consumption photography and the low power consumption photography are prohibited, and for example, it is displayed in red color. In addition to the battery state sign 13, the controller 101 may make the display 103 also display a message 13A: “All types of photography are unavailable due to insufficient remaining battery power. Charge the battery.” In this case, in response to pressing down a close button 13B by operation to the operation interface 102, the controller 101 hides the message 13A.

As described above, the first embodiment uses the two thresholds: the first threshold for allowing photographing of the first photography mode (high power consumption photography), and the second threshold for allowing photographing of the second photography mode that consumes less power than the first photography mode (low power consumption photography). Thus, the low power consumption photography can be performed although the remaining battery power is insufficient for performing the high power consumption photography.

In one example, when the remaining battery power is insufficient for performing the moving image photography, the still image photography can be performed on the condition that the remaining battery power is equal to or greater than the second threshold.

In another example, when the remaining battery power is insufficient for performing the non-cooperation photography, the device-cooperation photography can be performed on the condition that the remaining battery power is equal to or greater than the second threshold.

In another example, when the remaining battery power is insufficient for performing the high definition photography, the low definition photography can be performed on the condition that the remaining battery power is equal to or greater than the second threshold.

In yet another example, when the remaining battery power is insufficient for performing the high-frame-rate moving image photography, the low-frame-rate moving image photography can be performed on the condition that the remaining battery power is equal to or greater than the second threshold.

It is possible for the FPD cassette 3 to determine whether photographing of each of the first photography mode and the second photography mode is available, in accordance with whether the remaining battery power is equal to or greater than the first threshold or the second threshold.

It is also possible for the FPD cassette 3 to show information related to the result of determination using the two thresholds, in the display style corresponding to the determination result. Specifically, the range that the remaining battery power is in is easily recognized among the following ranges: the range in which each of the high power consumption photography and the low power consumption photography is available, the range in which the high power consumption photography is unavailable but the low power consumption photography is available, and the range in which both of the high power consumption photography and the low power consumption photography are unavailable.

It is also possible for the FPD cassette 3 to transmit the information related to the result of determination using the two thresholds, to the main body 1.

Thus, the main body 1 can notify the determination result, based on the information related to the determination result received from the FPD cassette 3. Notifying the determination result by the main body 1, which functions as the control device for controlling radiography performed by the FPD cassette 3, facilitates a user to recognize whether photographing of each photography mode is available.

### <Second Embodiment>

Next, a second embodiment using the present invention will be described.

The configuration of the second embodiment is the same as that described in relation to the first embodiment, and therefore, illustrations and descriptions thereof are omitted by referring to FIGS. 1 to 3. Hereinafter, the configuration and process characteristic of the second embodiment will be described.

In the first embodiment, the FPD cassette 3 determines whether photographing of each photography mode is available, based on the remaining battery power. On the other hand, in the second embodiment, the main body 1 determines whether photographing of each photography mode is available, based on the remaining battery power that is acquired from the FPD cassette 3.

### (Configuration of Radiography System)

The controller 301 of the FPD cassette 3 measures the amount of power remaining in the battery 307.

The controller 301 transmits information of the remaining battery power, which is measured with respect to the battery 307, to the main body 1 functioning as the control device, via the wireless communicator 304. That is, the controller 301 also functions as a transmission controller. For example, the controller 301 makes a measurement of the remaining battery power and a transmission to the main body 1, such as when the power supply is turned on by the power switch 305, or when photographing is finished once.

The storage 104 of the main body 1 stores a first threshold and a second threshold of the amount of power remaining in the battery. The first threshold allows photographing of a first photography mode. The second threshold allows photographing of a second photography mode that consumes less power than the first photography mode. In the second embodiment, it is not necessary for the FPD cassette 3 to store the first threshold and the second threshold.

In one example, the first photography mode is a moving image photography mode, whereas the second photography mode is a still image photography mode.

The first photography mode is also a photography mode in which the FPD cassette 3 performs photographing without cooperation with the radiation source 2 (non-cooperation photography mode), whereas the second photography mode is a photography mode in which the FPD cassette 3 performs photographing in cooperation with the radiation source 2 (device-cooperation photography mode).

The first photography mode is also a high definition photography mode, whereas the second photography mode is also a low definition photography mode with a resolution lower than that of the high definition photography mode.

In addition, the first photography mode is a high-frame-rate moving image photography mode, whereas the second photography mode is a low-frame-rate moving image photography mode in which the frame rate is lower than that of the high-frame-rate moving image photography mode.

The controller 101 of the main body 1 determines that photographing of the first photography mode is available in the case in which the remaining battery power is equal to or greater than the first threshold. The controller 101 also determines that photographing of the second photography mode is available but photographing of the first photography mode is unavailable in the case in which the remaining battery power is equal to or greater than the second threshold but is less than the first threshold. The controller 101 also determines that photographing of the second photography mode is unavailable in the case in which the remaining battery power is less than the second threshold. That is, the controller 101 also functions as a determination unit.

The first threshold is greater than the second threshold, and therefore, photographing of the second photography mode is also available in the case in which the remaining battery power is equal to or greater than the first threshold.

Conversely, the second threshold is smaller than the first threshold, and therefore, photographing of the first photography mode is also unavailable in the case in which the remaining battery power is less than the second threshold.

The controller 101 notifies the result of determining whether photographing of each photography mode is available, based on the remaining battery power. That is, the controller 101 also functions as a notification unit.

### (Operation of Radiography System)

Next, operation of the radiography system 100 of the second embodiment will be described.

FIG. 12 is a flowchart illustrating a second process of determining remaining battery power, which is executed by the controller 101 of the main body 1. The second process of determining remaining battery power is executed by cooperation of the controller 101 and the program stored in the storage 104.

First, the controller 101 of the main body 1 receives information of the remaining battery power (amount of stored power) from the FPD cassette 3, via the first communication unit 105a (step S11).

Next, the controller 101 determines whether the remaining battery power shown by the received information is equal to or greater than the first threshold, which is stored in the storage 104 (step S12).

In the case in which the remaining battery power is equal to or greater than the first threshold (step S12; YES), the controller 101 determines that photographing of the first photography mode (high power consumption photography) is available (step S13). The first threshold is greater than the second threshold, and therefore, photographing of the second photography mode (low power consumption photography) is also available in the case in which the remaining battery power is equal to or greater than the first threshold.

Then, the controller 101 notifies the state that each of the high power consumption photography and the low power consumption photography is available (step S14). For example, the controller 101 makes the display 103 display the battery state sign 11 and the message 11A illustrated in FIG. 9, in the panel state display area 1032 of the operation screen 1031 (refer to FIG. 8).

In the case in which the remaining battery power is less than the first threshold in step S12 (step S12; NO), the controller 101 determines whether the remaining battery power is equal to or greater than the second threshold, which is stored in the storage 104 (step S15).

In the case in which the remaining battery power is equal to or greater than the second threshold (step S15; YES), the controller 101 determines that the high power consumption photography is unavailable but the low power consumption photography is available (step S16).

Then, the controller 101 notifies the state that the high power consumption photography is prohibited but the low power consumption photography is available (step S17). For example, the controller 101 makes the display 103 display the battery state sign 12 and the message 12A illustrated in FIG. 10, in the panel state display area 1032 of the operation screen 1031 (refer to FIG. 8).

In the case in which the remaining battery power is less than the second threshold in step S15 (step S15; NO), the controller 101 determines that the low power consumption photography is unavailable (step S18).

Then, the controller 101 notifies the state that both of the high power consumption photography and the low power consumption photography are prohibited (step S19). For example, the controller 101 makes the display 103 display the battery state sign 13 and the message 13A illustrated in FIG. 11, in the panel state display area 1032 of the operation screen 1031 (refer to FIG. 8).

After step S14, S17, or S19 is performed, the second process of determining remaining battery power is completed.

As described above, the second embodiment uses the two thresholds: the first threshold for allowing photographing of the first photography mode (high power consumption photography), and the second threshold for allowing photographing of the second photography mode that consumes less power than the first photography mode (low power consumption photography). Thus, the low power consumption photography can be performed although the remaining battery power is insufficient for performing the high power consumption photography.

It is possible for the main body 1 to determine whether photographing of each of the first photography mode and the second photography mode is available, in accordance with whether the remaining battery power, which information is received from the FPD cassette 3, is equal to or greater than the first threshold or the second threshold.

Notifying the determination result by the main body 1 facilitates a user to recognize whether photographing of each photography mode is available.

Note that each of the foregoing embodiments describes an example of the radiography system according to the present invention, and the embodiments are not limited thereto. Details of the structure and details of the operation of each device that constitutes the system can also be modified or altered within a range not departing from the gist of the present invention, as appropriate.

The first threshold and the second threshold are stored in advance in the storage 303 of the FPD cassette 3 or the storage 104 of the main body 1 in the above-described embodiments. However, for example, a threshold that is necessary to perform photographing may be set based on the amount of image data estimated from photographing conditions in radiography of each photography mode. Examples of the photographing conditions include the number of photographic images to be obtained and the number of pixels of the FPD cassette 3 to be used.

In the second embodiment, the main body 1 determines whether photographing of each photography mode is available, by using the first threshold and the second threshold preliminarily stored in the storage 104. However, the FPD cassette 3 may transmit information of the first threshold and the second threshold in addition to the remaining battery power, to the main body 1, and the main body 1 may determine whether photographing of each photography mode is available, by using the first threshold and the second threshold, which information is received from the FPD cassette 3.

The first embodiment and the second embodiment are described by using two photography modes that consume different amounts of power. However, three or more types of photography modes having different power consumption may be used by setting three or more resolution levels or frame rates, and a threshold for allowing/prohibiting photographing may be specified with respect to each photography mode.

In each of the above-described embodiments, the first threshold is used as a threshold for determining allowing/prohibiting of photographing of a photography mode in which the amount of power consumption is great and the maximum amount of power consumption for performing photographing once is known in advance, such as the moving image photography mode or the high definition photography mode. However, in a case in which the end time of photographing is uncertain due to the photographing time depending on a user, such as in fluorography, the first threshold may be a threshold for preliminarily notifying a user of an alert such as “Remaining time available for photography is X seconds.”

A computer-readable medium that stores the program for executing each process is not limited to the examples described above. In addition, a carrier wave may be used as a medium that provides data of the program via a communication line.

Although embodiments of the present invention have been described and illustrated in detail, the disclosed embodiments are made for purposes of illustration and example only and not limitation. The scope of the present invention should be interpreted by terms of the appended claims.

What is claimed is:

1. A radiographic imaging device that obtains a radiographic image, comprising:
a battery that drives the radiographic imaging device;
a first hardware processor that measures an amount of power remaining in the battery; and
a storage that stores a first threshold and a second threshold of the amount of power remaining in the battery, the first threshold used to allow photographing of a first photography mode, the second threshold used to allow photographing of a second photography mode that consumes less power than the first photography mode.

2. The radiographic imaging device according to claim 1, wherein the first photography mode is a moving image photography mode, and the second photography mode is a still image photography mode.

3. The radiographic imaging device according to claim 1, wherein the first photography mode is a photography mode in which the radiographic imaging device performs photographing without cooperation with a radiation irradiation device, and the second photography mode is a photography mode in which the radiographic imaging device performs photographing in cooperation with the radiation irradiation device.

4. The radiographic imaging device according to claim 1, wherein the first photography mode is a high definition photography mode, and the second photography mode is a low definition photography mode with a resolution lower than a resolution of the high definition photography mode.

5. The radiographic imaging device according to claim 1, wherein the first photography mode is a high-frame-rate moving image photography mode, and the second photography mode is a low-frame-rate moving image photography mode in which a frame rate is lower than a frame rate of the high-frame-rate moving image photography mode.

6. The radiographic imaging device according to claim 1, wherein the first hardware processor determines that photographing of the first photography mode is available in a case in which the amount of power remaining in the battery is equal to or greater than the first threshold, determines that photographing of the second photography mode is available but photographing of the first photography mode is unavailable in a case in which the amount of power remaining in the battery is equal to or greater than the second threshold but is less than the first threshold, and determines that photographing of the second photography mode is unavailable in a case in which the amount of power remaining in the battery is less than the second threshold.

7. The radiographic imaging device according to claim 6, further comprising a display that displays information related to result of determination performed by the first hardware processor,
the first hardware processor making the display show a different display depending on the result of determination.

8. The radiographic imaging device according to claim 6, wherein the first hardware processor transmits the information related to the result of determination to a control device that controls radiography performed by the radiographic imaging device.

9. A radiography system comprising:
the radiographic imaging device according to claim 8; and
the control device,
the control device including a second hardware processor that notifies the result of determination, based on the information related to the result of determination, which is received from the radiographic imaging device.

10. A radiography system comprising:
a radiographic imaging device that obtains a radiographic image; and
a control device that controls radiography performed by the radiographic imaging device, the radiographic imaging device including:
a battery that drives the radiographic imaging device; and
a first hardware processor that measures an amount of power remaining in the battery and transmits information of the amount of power remaining in the battery to the control device,
the control device including:
a storage that stores a first threshold and a second threshold of the amount of power remaining in the battery, the first threshold used to allow photographing of a first photography mode, the second threshold used to allow photographing of a second photography mode that consumes less power than the first photography mode; and
a second hardware processor that:
determines that photographing of the first photography mode is available in a case in which the amount of power remaining in the battery is equal to or greater than the first threshold;
determines that photographing of the second photography mode is available but photographing of the first photography mode is unavailable in a case in which the amount of power remaining in the battery is equal to or greater than the second threshold but is less than the first threshold;
determines that photographing of the second photography mode is unavailable in a case in which the amount of power remaining in the battery is less than the second threshold; and
notifies result of the determination.

11. The radiography system according to claim 10, wherein the first photography mode is a moving image photography mode, and the second photography mode is a still image photography mode.

12. The radiography system according to claim 10, wherein the first photography mode is a photography mode in which the radiographic imaging device performs photographing without cooperation with a radiation irradiation device, and the second photography mode is a photography mode in which the radiographic imaging device performs photographing in cooperation with the radiation irradiation device.

13. The radiography system according to claim 10, wherein the first photography mode is a high definition photography mode, and the second photography mode is a low definition photography mode with a resolution lower than a resolution of the high definition photography mode.

14. The radiography system according to claim 10, wherein the first photography mode is a high-frame-rate moving image photography mode, and the second photography mode is a low-frame-rate moving image photography mode in which a frame rate is lower than a frame rate of the high-frame-rate moving image photography mode.
